(19) United States
(12) Patent Application Publication
(10) Pub. No.: US 2026/0298949 A1
(43) Pub. Date: Oct. 1, 2026

(54) PROTEIN FREE SOLUTION FOR STABILIZING IMMUNOASSAY CALIBRATORS

(71) Applicant: BECKMAN COULTER, INC., Brea, CA (US)

(72) Inventors: Cliff Briggs, Chaska, MN (US); Michael Salvati, Chaska, MN (US); Ian Sinclair, Chaska, MN (US); Jeff Todtleben, Chaska, MN (US); Ryan Workman, Chaska, MN (US); Paul Wynveen, Chaska, MN (US)

(21) Appl. No.: 19/475,919

(22) PCT Filed: Apr. 22, 2024
(86) PCT No.: PCT/US2024/025689

§ 371 Date: Oct. 16, 2025

Related U.S. Application Data
(60) Provisional application No. 63/462,441, filed on Apr. 27, 2023.

(51) Int. Cl. G01N 33/74; G01N 33/53; G01N 33/96

(52) U.S. Cl. CPC G01N 33/74; G01N 33/5306; G01N 33/96; G01N 2333/58

(57) ABSTRACT

Disclosed and claimed are compositions containing a calibrator and a stabilizer, wherein the stabilizer includes Diethylaminoethyl-(“DEAE”) dextran; D-Sorbitol, wherein the D-Sorbitol and DEAE-dextran are in a weight ratio from about 1:1 to about 5:2, a buffer; and a non-ionic surfactant; wherein the stabilizer is essentially free of bovine serum albumin; and wherein the calibrator is stabilized under frozen storage conditions, and remains stable when thawed and kept at temperatures of from about 2° C. to about 10° C. Also disclosed are methods of use of the claimed compositions and kits comprising the claimed compositions.

### RELATED APPLICATIONS

The present patent application claims the priority benefit of U.S. Provisional Patent Application Ser. No. 63/462,441, filed Apr. 27, 2023, the content of which is hereby incorporated by reference in its entirety into this disclosure.

### BACKGROUND

Immunoassays are widely used in clinical and research settings for the detection and quantification of target molecules in various samples (e.g., serum, plasma, whole blood). Before a sample in an immunoassay can be evaluated, calibration is required. The process of calibration generally involves using an immunoassay calibrator (e.g., a recombinant protein or synthetic peptide) as a reference point for quantifying the amount of a target molecule in a sample. Often, a series of dilutions of the calibrator of known concentrations are prepared. Each of the dilutions is measured using the immunoassay, and the resulting signals are plotted against the corresponding known concentrations. A curve generated from the plot is then used to correlate the signal produced by the sample to the concentration of the target molecule in the sample.

The stability of a calibrator is critical to assay performance because changes in the concentration or quality of the calibrator can impact the accuracy and precision of the assay. The calibrator must be stable over the entire range of concentrations used in the assay, as degradation or loss of activity may produce inaccurate assay results and potentially incorrect clinical decisions.

Therefore, a need exists for a stable calibrator product capable of calibrating an immunoassay. However, achieving and maintaining stability of a calibrator peptide or protein can be challenging. Over time, exposure to temperature, humidity and light can cause denaturation, degradation, and aggregation of the calibrator, resulting in a loss of activity. In commercial settings, it is common for immunoassay customers to store calibrators for extended periods, ranging from several weeks to months, before using them. Similarly, calibrators may also be stored in a manufacturing or distribution facility for an extended period of time before being shipped to customers.

To extend shelf life, calibrators are often frozen. However, in many cases, calibrators thaw unintentionally during the shipment process. Additionally, customers may choose to freeze a calibrator between uses. Freeze-thaw cycles can cause mechanical damage to the structure of a calibrator protein or peptide, increasing the likelihood of denaturation, degradation, aggregation, and loss of activity.

Some calibrator proteins and peptides—e.g., Natriuretic peptides (e.g., BNP, NT-pro BNP), are relatively unstable. As a result, their use in calibrator formulations for commercialized assays most often requires them to be freeze-dried (lyophilization). Lyophilized calibrators are often more expensive than liquid calibrators due to the additional processing and packaging steps involved. Further, reconstituting a lyophilized calibrator adds to the total time required to perform an assay, as compared to a liquid calibrator. Once reconstituted, there is a short timeframe to the use the calibrator, after which it must are either discarded, held short term (typically <24 hours at 2-8° C.), or in some cases, refrozen for longer term storage. Frozen calibrators must then be thawed before any subsequent use, further adding time to the operation.

Therefore, a need exists for a matrix that provides stability for calibrator proteins-including Natriuretic peptides (e.g., BNP, NTproBNP)—at refrigerated and warmer storage temperatures and through multiple freeze-thaw cycles. Elimination of lyophilization requirements would significantly reduce calibrator manufacturing processing and costs and product performance variability due to reconstitution.

### BRIEF SUMMARY

The present disclosure relates to a bovine serum albumin (BSA)-free matrix for stabilizing a calibrator or control, e.g., for use in an immunoassay.

In many cases, calibrator formulations comprise a matrix that closely mimics a sample being tested (e.g., serum, plasma, whole blood), and a calibrator (e.g., a protein or peptide used to establish a concentration of an analyte). Such conventional calibrator formulations generally include bovine serum albumin (BSA), a compound for preventing fungal and bacterial growth (e.g., Proclin 300 or Sodium Azide), a PH neutral buffer, and a detergent or a surfactant. The BSA protein stabilizes the calibrator such that the calibrator does not aggregate, clump together, or otherwise interact with its container. BSA is also commonly chosen because it provides an environment that more closely mimics a human plasma sample as compared to other compounds. However, proteins such as BSA can affect assay performance. Calibrator solutions containing BSA, when in use with stability-sensitive peptides or proteins, further require protease inhibitors, which also add to manufacturing costs. Additionally, these solutions are often provided to the end user as a lyophilized composition and require reconstitution.

The calibrator matrix of the present invention is free, or substantially free, of BSA. In certain embodiments, the calibrator matrix of the present invention is free, or substantially free, of protein (apart from the calibrator). In some cases, the calibrator matrix of the present invention does not comprise a protease inhibitor because it does not comprise BSA which may contain proteases capable of inducing calibrator degradation. In some cases, the calibrator matrix of the present invention can be manufactured for a lower cost than a conventional calibrator formulation because it does not contain protease inhibitors.

The calibrator matrix of the present invention is also capable of stabilizing a calibrator protein—including a natriuretic peptide or protein—without being lyophilized. In some cases, the calibrator matrix of the present invention is advantageous relative to lyophilized calibrator formulations because it does not require reconstitution, which is associated with more complex manufacturing processes and increased customer workflow as compared to a liquid calibrator solution.

One aspect of the present disclosure is a composition including at least one calibrator; and a stabilizer, wherein the stabilizer includes Diethylaminoethyl-(“DEAE”) dextran; D-Sorbitol, wherein the D-Sorbitol and DEAE-dextran are in a weight ratio from about 1:1 to about 5:2, a buffer; and a non-ionic surfactant; wherein the stabilizer is essentially free of bovine serum albumin; and wherein the calibrator is stabilized under frozen storage conditions, and remains stable when thawed and kept at temperatures of from about 2° C. to about 10° C.

In an aspect, the calibrator remains stable under frozen conditions for at least about 1 month, alternatively at least about 2 months, alternatively at least about 3 months, alternatively at least about 4 months, alternatively at least about 5 months, alternatively at least about 6 months, alternatively at least about 7 months, alternatively at least about 8 months, alternatively at least about 9 months, alternatively at least about 10 months, alternatively at least about 11 months, alternatively at least about 12 months.

In an aspect, the calibrator remains stable when thawed and kept at temperatures of from about 2° C. to about 10° C. for at least about 24 hours, alternatively at least about 3 days, alternatively at least about 1 week, alternatively at least about 2 weeks, alternatively at least about 3 weeks, alternatively at least about 4 weeks, alternatively at least about 5 weeks, alternatively at least about 6 weeks, alternatively at least about 7 weeks, alternatively at least about 8 weeks, alternatively at least about 9 weeks.

In another aspect, the calibrator remains stable after at least one freeze-thaw cycle, alternatively after at least two freeze-thaw cycles, alternatively after at least three freeze-thaw cycles, alternatively after at least four freeze-thaw cycles, alternatively after at least five freeze-thaw cycles, alternatively after at least six freeze-thaw cycles, alternatively after at least seven freeze-thaw cycles.

In an aspect, the D-Sorbitol and DEAE-dextran are in a weight ratio from about 1:1 to about 2:3, alternatively from about 3:1 to about 5:2, alternatively from about 1.8:1 to about 2.2:1, alternatively about 1.9:1, alternatively about 2:1, alternatively about 2:3, alternatively about 5:2.

In an aspect, the stabilizer includes about 0.5 wt. % to about 4 wt. % D-Sorbitol, alternatively about 1 wt. % to about 3.5 wt. % D-Sorbitol, alternatively about 1.5 wt. % to about 2.5 wt. % D-Sorbitol, alternatively about 1.6 wt. % D-Sorbitol, alternatively about 1.7 wt. % D-Sorbitol, alternatively about 1.8 wt. % D-Sorbitol, alternatively about 1.9 wt. % D-Sorbitol, alternatively about 2.0 wt. % D-Sorbitol, alternatively about 2.1 wt. % D-Sorbitol, alternatively about 2.2 wt. % D-Sorbitol, alternatively about 2.3 wt. % D-Sorbitol, alternatively about 2.4 wt. % D-Sorbitol, or alternatively about 2.5 wt. % D-Sorbitol.

In an aspect, the stabilizer includes about 0.25 wt. % to about 2.5 wt. % DEAE-dextran, alternatively about 0.5 wt. % to about 2 wt. % DEAE-dextran, alternatively about 0.5 wt. % to about 1.5 wt. % DEAE-dextran, alternatively about 0.6 wt. % DEAE-dextran, alternatively about 0.7 wt. % DEAE-dextran, alternatively about 0.8 wt. % DEAE-dextran, alternatively about 0.9 wt. % DEAE-dextran, alternatively about 1.0 wt. % DEAE-dextran, alternatively about 1.1 wt. % DEAE-dextran, alternatively about 1.2 wt. % DEAE-dextran, alternatively about 1.3 wt. % DEAE-dextran, or alternatively about 1.4 wt. % DEAE-dextran.

In an aspect, the buffer is a citrate buffer, an acetate buffer, a phosphate buffer, or combinations thereof. In another aspect, the buffer is selected from the group consisting of citrate, citric acid monohydrate, sodium acetate, potassium acetate, sodium phosphate, or combinations thereof.

In an aspect, the stabilizer comprises at least about 0.0025 M buffer, alternatively at least about 0.001 M buffer, alternatively at least about 0.002 M buffer, alternatively at least about 0.003 M buffer, alternatively at least about 0.004 M buffer, alternatively at least about 0.005 M buffer, alternatively at least about 0.006 M buffer, or alternatively at least about 0.007 M buffer.

In an aspect, the non-ionic surfactant is a secondary alcohol ethoxylate, a polyether, a poly (ethylene glycol) derivative, or combinations thereof. In another aspect, the non-ionic surfactant is selected from the group consisting of Alkyl polyglycoside, Cetomacrogol 1000, Cetostearyl alcohol, Cetyl alcohol, Cocamide DEA, Cocamide MEA, Decyl glucoside, Decyl polyglucose, Glycerol monostearate, IGEPAL CA-630, Isoceteth-20, Lauryl glucoside, Maltoside, Monolaurin, Mycosubtilin, Narrow-range ethoxylate, Nonidet P-40, Nonoxynol-9, Nonoxynols, NP-40, Octaethylene glycol monododecyl ether, N-Octyl beta-D-thioglucopyranoside, Octyl glucoside, Oleyl alcohol, Pentaethylene glycol monododecyl ether, Polidocanol, Poloxamer, Poloxamer 407, Polyethoxylated tallow amine, Polyglycerol polyricinoleate, Polysorbate, Polysorbate 20, Polysorbate 80, Sorbitan, Sorbitan monolaurate, Sorbitan monostearate, Sorbitan tristearate, Stearyl alcohol, Surfactin, Tergitol, Triton X-100, Tween 80, and combinations thereof.

In an aspect, the stabilizer comprises about 0.25 wt. % to about 2.5 wt. % of the non-ionic surfactant, alternatively about 0.5 wt. % to about 2 wt. % of the non-ionic surfactant, alternatively about 0.095 wt. % to about 1.5 wt. % of the non-ionic surfactant, alternatively about 0.10 wt. % of the non-ionic surfactant, alternatively about 0.101 wt. % of the non-ionic surfactant, alternatively about 0.102 wt. % of the non-ionic surfactant, alternatively about 0.103 wt. % of the non-ionic surfactant, alternatively about 0.104 wt. % of the non-ionic surfactant, alternatively about 0.105 wt. % of the non-ionic surfactant, alternatively about 0.2 wt. % of the non-ionic surfactant, alternatively about 0.3 wt. % of the non-ionic surfactant, alternatively about 0.4 wt. % of the non-ionic surfactant, alternatively about 0.5 wt. % of the non-ionic surfactant, alternatively about 0.6 wt. % of the non-ionic surfactant, alternatively about 0.7 wt. % of the non-ionic surfactant, alternatively about 0.8 wt. % of the non-ionic surfactant, alternatively about 0.9 wt. % of the non-ionic surfactant, alternatively about 1.0 wt. % of the non-ionic surfactant, alternatively about 1.1 wt. % of the non-ionic surfactant, alternatively about 1.2 wt. % of the non-ionic surfactant, alternatively about 1.3 wt. % of the non-ionic surfactant, alternatively about 1.4 wt. % of the non-ionic surfactant, alternatively about 1.5 wt. % of the non-ionic surfactant, or alternatively about 0.095 wt. % to about 0.105 wt. % non-ionic surfactant.

In an aspect, the stabilizer is free of bovine serum albumin. In another aspect, the composition is essentially free of bovine serum albumin, alternatively free of bovine serum albumin. In another aspect, the composition is essentially free of protease inhibitors, preferably free of protease inhibitors.

In an aspect, the composition is a liquid or a solution. In an aspect, the composition has a pH of from about 3.5 to about 6.5, alternatively about 3.5 to about 4.5, alternatively about 4.5 to about 5, alternatively about 3.5, alternatively about 4, alternatively about 4.5, alternatively about 5, alternatively about 5.5, alternatively about 6.5.

In an aspect, the calibrator is an immunoassay calibrator. In an aspect, the calibrator is substantially similar to a target analyte. In another aspect, the target analyte is a biomarker or a diagnostic analyte. In an aspect, the calibrator is a protein, peptide, or amino acid. In an aspect, the calibrator is recombinantly or synthetically produced. In an aspect, the calibrator comprises a post-translational modification. In another aspect, the post-translational modification is glycosylation, phosphorylation, or acetylation.

In an aspect, the calibrator is a natriuretic peptide or natriuretic protein. In an aspect, the calibrator is a recombinantly produced and glycosylated N-Terminal Pro-B-Type Natriuretic peptide (NT-proBNP). In an aspect, the calibrator is a synthetically produced NT-proBNP. In an aspect, the calibrator is a recombinant B-type natriuretic peptide (BNP).

One aspect of the disclosure is a method for quantitative assay calibration, the method including analyzing at least one composition of the preceding claims using an immunoassay, wherein the analyzing includes detecting the presence, absence, and/or quantity of the calibrator in the at least one composition, and generating a corresponding set of values; and wherein the corresponding set of values is used to generate a calibration curve.

In an aspect, the method includes analyzing at least two compositions of any one of the preceding claims; alternatively at least three compositions of any one of the preceding claims; alternatively at least four compositions of any one of the preceding claims; alternatively at least five compositions of any one of the preceding claims; alternatively at least six compositions of any one of the preceding claims and wherein each composition comprises a different concentration of the calibrator.

In an aspect, the calibration curve is a non-linear calibration curve. In another aspect, the non-linear calibration curve is used to quantify a target analyte. In another aspect, the non-linear calibration curve includes at least two values, wherein one value corresponds to the concentration of calibrator in the first composition and one value corresponds to the concentration of calibrator in the second composition. In another aspect, the non-linear calibration curve includes at least three values, wherein one value corresponds to the concentration of calibrator in the third composition. In another aspect, the non-linear calibration curve includes at least four values, wherein one value corresponds to the concentration of calibrator in the fourth composition. In another aspect, the non-linear calibration curve includes at least five values, wherein one value corresponds to the concentration of calibrator in the fifth composition. In another aspect, the non-linear calibration curve includes at least six values, wherein one value corresponds to the concentration of calibrator in the sixth composition.

In an aspect, the method is conducted using an analyzer. In another aspect, the analyzer is an immunoassay analyzer. In another aspect, the analyzer is a high-throughput analyzer.

In an aspect of the method, the calibrator is a natriuretic peptide or natriuretic protein. In another aspect of the method, the calibrator is a recombinantly produced and glycosylated N-Terminal Pro-B-Type Natriuretic peptide (NT-proBNP). In another aspect of the method, the calibrator is a synthetically produced NT-proBNP. In another aspect of the method, the calibrator is a recombinant B-type natriuretic peptide (BNP).

One aspect of the disclosure is a kit including at least a first composition according to any one of the preceding paragraphs and a second composition according to any one of the preceding paragraphs and instructions for use, wherein the immunoassay calibrator concentration in the first composition and the second composition are different.

In an aspect, the kit further includes a third composition according to any one of the preceding paragraphs, wherein the immunoassay calibrator concentration in the third composition is different from the immunoassay calibrator concentration in the first composition and the second composition.

In an aspect, the kit further includes a fourth composition according to any one of the preceding paragraphs, wherein the immunoassay calibrator concentration in the fourth composition is different from the immunoassay calibrator concentration in the first composition, the second composition, and the third composition.

In an aspect, the kit further includes a fifth composition according to any one of the preceding paragraphs, wherein the immunoassay calibrator concentration in the fifth composition is different from the immunoassay calibrator concentration in the first composition, the second composition, the third composition, and the fourth composition.

In an aspect, the kit further includes a sixth composition according to any one of the preceding paragraphs, wherein the calibrator concentration in the sixth composition is different from the calibrator concentration in the first composition, the second composition, the third composition, the fourth composition, and the fifth composition.

In an aspect of the kit, the calibrator concentration in the third composition, fourth composition, fifth composition, or sixth composition is zero.

In an aspect of the kit, the first composition, second composition, third composition, fourth composition, fifth composition, and/or the sixth composition are in separate units, vials, or containers.

In an aspect, the kit further includes an antigen of the calibrator. In another aspect, the antigen is an NT-proBNP or BNP antigen.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

Various aspects of the present disclosure will now be described, by way of example only, with reference to the attached Figures, wherein:

FIG. 1 depicts a linear model for percent (%) change in calibrator concentration versus freeze-thaw cycle for a composition according to an aspect of this disclosure containing 55 ng/L of NT-proBNP.

FIG. 2 depicts a linear model for percent (%) change in calibrator concentration versus freeze-thaw cycle for a composition according to an aspect of this disclosure containing 467 ng/L of NT-proBNP.

FIG. 3 depicts a linear model for percent (%) change in calibrator concentration versus freeze-thaw cycle for a composition according to an aspect of this disclosure containing 3,958 ng/L of NT-proBNP.

FIG. 4 depicts a linear model for percent (%) change in calibrator concentration versus freeze-thaw cycle for a composition according to an aspect of this disclosure containing 21,622 ng/L of NT-proBNP.

FIG. 5 depicts a linear model of short-term stability for a composition according to an aspect of this disclosure containing 60 ng/L of NT-proBNP and stored at refrigerated temperatures.

FIG. 6 depicts a linear model of short-term stability for a composition according to an aspect of this disclosure containing 200 ng/L of NT-proBNP and stored at refrigerated temperatures.

FIG. 7 depicts a linear model of short-term stability for a composition according to an aspect of this disclosure containing 650 ng/L of NT-proBNP and stored at refrigerated temperatures.

FIG. 8 depicts a linear model of short-term stability for a composition according to an aspect of this disclosure containing 2200 ng/L of NT-proBNP and stored at refrigerated temperatures.

FIG. 9 depicts a linear model of short-term stability for a composition according to an aspect of this disclosure containing 7000 ng/L of NT-proBNP and stored at refrigerated temperatures.

FIG. 10 depicts a linear model of short-term stability for a composition according to an aspect of this disclosure containing 23000 ng/L of NT-proBNP and stored at refrigerated temperatures.

FIG. 11 depicts a linear model of long-term stability for a composition according to an aspect of this disclosure containing 60 ng/L of NT-proBNP and stored at frozen temperatures.

FIG. 12 depicts a linear model of long-term stability for a composition according to an aspect of this disclosure containing 200 ng/L of NT-proBNP and stored at frozen temperatures.

FIG. 13 depicts a linear model of long-term stability for a composition according to an aspect of this disclosure containing 650 ng/L of NT-proBNP and stored at frozen temperatures.

FIG. 14 depicts a linear model of long-term stability for a composition according to an aspect of this disclosure containing 2200 ng/L of NT-proBNP and stored at frozen temperatures.

FIG. 15 depicts a linear model of long-term stability for a composition according to an aspect of this disclosure containing 7000 ng/L of NT-proBNP and stored at frozen temperatures.

FIG. 16 depicts a linear model of long-term stability for a composition according to an aspect of this disclosure containing 23000 ng/L of NT-proBNP and stored at frozen temperatures.

### DETAILED DESCRIPTION

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Herein, “up to” a number (for example, up to 50) includes the number (for example, 50).

The term “in the range” or “within a range” (and similar statements) includes the endpoints of the stated range.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine). The term, as used herein, refers to proteins, polypeptides, and peptides of any size, structure, or function. Polypeptides include gene products, naturally occurring polypeptides, recombinant polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer, or tetramer. They can also comprise single chain or multichain polypeptides. Most commonly, disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, 10, 15, 20, 25, 30, 35, 40, 45, or 50 amino acids long.

The terms “recombinant protein” and “recombinantly produced protein” are used interchangeably herein to refer to proteins that are formed by transfecting a host cell with a gene of interest. The gene of interest is isolated and closed into an expression vector. Several organisms are currently used for recombinant protein production, for example, Escherichia coli. The terms “synthetic protein” and “synthetically produced proteins” are used interchangeably herein to refer to proteins that are artificially manufactured. Both recombinant protein and synthetic protein substantially mimic the structure, function, and composition of naturally occurring proteins.

The invention is defined in the claims. However, below is a non-exhaustive listing of non-limiting exemplary aspects. Any one or more of the features of these aspects may be combined with any one or more features of another example, embodiment, or aspect described herein.

One aspect of the disclosure is a composition that includes at least one calibrator or control (e.g., an immunoassay calibrator, a control protein, or a control peptide) and a stabilizer. In some aspects, the composition comprises a BSA-free matrix for stabilizing the calibrator or control. In some instances, the composition comprises a protein-free matrix (apart from the calibrator or control) for stabilizing the calibrator or control.

The calibrator may be an immunoassay calibrator. Many immunoassay formats, e.g., enzyme-linked immunoassays, fluorescent immunoassays, chemiluminescent immunoassays, are well-known in the art and have been widely used for many years in clinical diagnostics and research applications. Skilled artisans will be familiar with the principles and techniques of these immunoassay formats, as well as the reagents used in these assays, including the calibrator. In many cases, a calibrator is a protein or peptide substantially similar to a target analyte, meaning that the calibrator and target analyte have similar structures and similar properties. This substantial similarity allows the calibrator to be used as a reference to determine an amount of the target analyte.

The calibrator may be a protein, peptide, or amino acid. In some instances, the calibrator is recombinantly produced. In some instances, the calibrator is synthetically produced. In some instances, the calibrator comprises one or more post-translational modifications to enhance its similarity to the target analyte, such as glycosylation, phosphorylation, or acetylation.

In most cases, the calibrator is used to calibrate the assay to establish a reference curve to determine the concentration of a target analyte. The calibrator is added to the assay in known concentrations and the signal produced by the assay at each concentration is measured. The measured signals are used to generate a standard curve or reference curve that relates the signal intensity to the calibrator concentration. To fit a calibrator curve to the measured signal, suitable mathematical functions known in the art (e.g., linear, quadratic, exponential) are used. Once the reference curve is created, a sample (e.g., a blood sample from a patient) is added to the assay and the signal produced by the assay is measured and compared to the reference curve. The reference curve may be then used to relate the signal generated by the assay to the amount of the target analyte.

The calibrator can be used in various types of immunoassays to construct a reference for the quantitation of a target analyte, such as a biomarker or a diagnostic analyte. The biomarker or diagnostic analyte may be for various diseases and/or disorders, including cardiac diseases, infectious diseases, oncological disorders, endocrine disorders, autoimmune diseases, neurological diseases, allergies, asthma, and combinations thereof. In some instances, the biomarker or diagnostic analyte can be used to diagnose any of the following diseases or disorders: myocardial infarction, heart failure (e.g., acute heart failure, chronic heart failure), lupus, rheumatoid arthritis, cancer (e.g., breast cancer, colorectal cancer, lung cancer, prostate cancer, ovarian cancer), blood viruses, diabetes, kidney diseases (e.g., chronic kidney disease, nephrotic syndrome), thyroid disorders, reproductive disorders, rheumatoid arthritis, inflammatory bowel disease, Alzheimer's disease, multiple sclerosis, sepsis, chronic obstructive pulmonary disease, and combinations thereof.

Non-limiting examples of suitable biomarkers include natriuretic peptides, N-Terminal Pro-B-Type Natriuretic peptide (NT-proBNP), B-type natriuretic peptide (BNP), alanine aminotransferase, alpha-fetoprotein, albumin, alkaline phosphatase, anti-müllerian hormone, apolipoprotein a, apolipoprotein a-1, apolipoprotein b, aspartate aminotransferase, B12, beta-amyloid, calcium, Bone-specific Alkaline Phosphatase, cancer antigen 15-3, cancer antigen 19-9, cancer antigen 125, cholesterol, Cytomegalovirus (CMV), c-reactive protein, Cortisol, C-peptide, creatine kinase myocardial band, creatine kinase, creatine kinase-myocardial, creatinine, cytokeratin 19 fragment, dehydroepiandrosterone sulfate, digoxin, direct bilirubin, erythropoietin, estradiol, ferritin, free/total prostate-specific antigen, follicle-stimulating hormone (FSH), fT4, gamma glutamyltransferase, glucose, hepatitis A, hepatitis A virus specific immunoglobulin G, Hepatitis B core antibody, Hepatitis B envelope antibody, high density lipoprotein-cholesterol, high density lipoprotein, human immunodeficiency virus, Hepatitis C, Hepatitis B, Hepatitis B Virus Surface Antigen, human chorionic gonadotropin, human growth hormone, Intrinsic factor antibodies, Interleukin-6 (IL-6), immunoglobulin G, inhibin A, insulin, luteinizing hormone (LH), low density lipoprotein, lipoprotein (a), luteinizing hormone, myoglobin, Neuron-specific Enolase, oestradiol, Parathyroid hormone, phosphate, potassium, Pro Prostate Specific Antigen, prostate specific antigen, prostate-specific membrane antigen, protein A, red blood cell, rheumatoid factor, serum pregnancy-associated plasma protein-A, procalcitonin, sex hormone binding globulin, sodium, tau proteins, testosterone, total bilirubin, total protein, triglyceride, triiodothyronine, thyroglobulin antibodies, Treponema pallidum antibody, troponin I, troponin T, thyroid stimulating hormone (TSH), Thyroxine (T4), Thyroid Peroxidase Antibodies, thyroid stimulating hormone, T3, tT3 urate, vitamin D, urea, and uric acid.

In exemplary aspects, the calibrator is a natriuretic peptide or natriuretic protein. In one embodiment, the calibrator is an NT-proBNP protein or peptide. The NT-proBNP protein may be synthetically or recombinantly produced. In one embodiment, the NT-proBNP protein is recombinantly produced and glycosylated. In another embodiment, the calibrator is a BNP protein or peptide.

In some instances, the calibrator may be present in compositions herein at a concentration of about 0-about 10 ng/L, about 10-about 20 ng/L, about 20-about 30 ng/L, about 30-about 40 ng/L, about 40-about 50 ng/L, about 50-about 60 ng/L, about 60-about 70 ng/L, about 70-about 80 ng/L, about 80-about 90 ng/L or about 90-about 100 ng/L. In some instances, the calibrator may be present in compositions herein at a concentration of about 0-about 100 ng/L, about 100-about 200 ng/L, about 200-about 300 ng/L, about 300-about 400 ng/L, about 400-about 500 ng/L, about 500-about 600 ng/L, about 600-about 700 ng/L, about 700-about 800 ng/L, about 800-about 900 ng/L or about 900-about 1,000 ng/L. In some instances, the calibrator may be present in compositions herein at a concentration of about 100-about 1000 ng/L, about 1000-about 2000 ng/L, about 2000-about 3000 ng/L, about 3000-about 4000 ng/L, about 4000-about 5000 ng/L, about 5000-about 6000 ng/L, about 6000-about 7000 ng/L, about 7000-about 8000 ng/L, about 8000-about 9000 ng/L or about 9000-about 10,000 ng/L. In some instances, the calibrator may be present in compositions herein at a concentration of about 1000-about 10,000 ng/L, about 10,000-about 20,000 ng/L, about 20,000-about 30,000 ng/L, about 30,000-about 40,000 ng/L, about 40,000-about 50,000 ng/L, about 50,000-about 60,000 ng/L, about 60,000-about 70,000 ng/L, about 70,000-about 80,000 ng/L, about 80,000-about 90,000 ng/L or about 90,000-about 100,000 ng/L. In some instances, the calibrator may be present in compositions herein at a concentration of about 0 ng/L, about 60 ng/L, about 300 ng/L, about 1200 ng/L, about 4000 ng/L, about 12000 ng/L, or about 40,000 ng/L.

The stabilizer may contain a dextran derivative, a sugar alcohol, a buffer, and a non-ionic surfactant. In a preferred embodiment, the stabilizer includes Diethylaminoethyl-(“DEAE”) dextran, D-Sorbitol, a buffer, and a non-ionic surfactant. The stabilizer is also essentially free, practically free, or free of protein. In non-limiting examples, the stabilizer is essentially free or practically free of bovine serum albumin (BSA). Essentially free or practically free means that the protein (or BSA) is present in an amount that is no more than about 0.5 wt. % of the stabilizer. In an exemplary aspect, the stabilizer is free of BSA. Free of protein (or BSA) means that no protein (or BSA) is detectable in the stabilizer. In some aspects, the composition is essentially free or practically free of BSA. In an exemplary aspect, the composition is free of BSA. In some instances, the stabilizer contains less than about 0.1 wt. % protein (or BSA). In some instances, the stabilizer contains less than about 0.01 wt. % protein (or BSA). In some instances, the stabilizer contains less than about 0.5 wt. %, about 0.4 wt. %, about 0.3 wt. %, about 0.2 wt. % or about 0.1 wt. % protein (or BSA). In some instances, the stabilizer contains less than about 0.1 wt. %, about 0.09 wt. %, about 0.08 wt. %, about 0.07 wt. %, about 0.06 wt. %., about 0.05 wt. %, about 0.04 wt. %, about 0.03 wt. %, about 0.02 wt. % or about 0.01 wt. % protein (or BSA).

BSA may contain proteases capable of inducing calibrator or control degradation. Additionally, BSA-containing compositions also contain preservatives to prevent fungal and bacterial growth. As such, a stabilizer and/or composition that is essentially free, practically free, or free of proteins, such as BSA, does not require protease inhibitors or preservatives. In a non-limiting example, the composition is essentially free, practically free, or free of protease inhibitors. In another non-limiting example, the composition is essentially free, practically free, or free of preservatives.

In some aspects, the dextran derivative is an aminodextran. In some instances, the sugar alcohol is D-sorbitol, mannitol, or ethylene glycol. In some aspects, the sugar alcohol and dextran derivative are in a weight ratio from about 1:1 to about 5:2. However, any suitable weight ratio may be used. In some non-limiting examples, the sugar alcohol and dextran derivative are in a weight ratio from about 1:1 to about 2:3, alternatively from about 3:1 to about 5:2, alternatively about 1.8:1 (9:5) to about 2.2:1 (11:5), alternatively about 1.9:1 (19:10), alternatively about 2:1, alternatively about 2:3, alternatively about 5:2. In a preferred embodiment, the sugar alcohol and dextran derivative are in a weight ratio from about 2:1.

In a preferred embodiment, the sugar alcohol is D-Sorbitol and the dextran derivative is DEAE dextran, and the D-Sorbitol and DEAE-dextran are in a weight ratio from about 1:1 to about 5:2. However, any suitable weight ratio may be used. In some non-limiting examples, the D-Sorbitol and DEAE-dextran are in a weight ratio from about 1:1 to about 2:3, alternatively from about 3:1 to about 5:2, alternatively about 1.8:1 (9:5) to about 2.2:1 (11:5), alternatively about 1.9:1 (19:10), alternatively about 2:1, alternatively about 2:3, alternatively about 5:2. In a preferred embodiment, the D-Sorbitol and DEAE-dextran are in a weight ratio from about 2:1.

The concentration of D-Sorbitol and DEAE-dextran in the stabilizer may also vary based on the desired weight ratio. For example, the stabilizer may include about 0.5 wt. % to about 4 wt. % D-Sorbitol, alternatively about 1 wt. % to about 3.5 wt. % % D-Sorbitol, alternatively about 1.5 wt. % to about 2.5 wt. % % D-Sorbitol. In some instances, the stabilizer may include about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, or about 2.5 wt. % D-Sorbitol. In some instances, the stabilizer may include about 2.0 wt. % D-Sorbitol.

In another example, the stabilizer may include about 0.25 wt. % to about 2.5 wt. % DEAE-dextran, alternatively about 0.5 wt. % to about 2 wt. % DEAE-dextran, alternatively about 0.5 wt. % to about 1.5 wt. % DEAE-dextran. In some instances, the stabilizer may include about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. % or about 1.5 wt. % DEAE-dextran. In some instances, the stabilizer may include about 1.0 wt. % DEAE-dextran.

In some aspects, the buffer is a citrate buffer, an acetate buffer, a phosphate buffer, or combinations thereof. For example, the buffer may be citrate, citric acid monohydrate, sodium acetate, potassium acetate, sodium phosphate, or combinations thereof. The buffer may also be Tris (Tromethamine). In a non-limiting example, the stabilizer includes at least about 0.005 M buffer. In some instances, the stabilizer includes at least 0.0025M buffer. In some instances, the stabilizer includes at least 0.001 M buffer. In some instances, the stabilizer includes about 0.005 M buffer. In some instances, the stabilizer includes from about 0.001-about 0.002 M, about 0.002-about 0.003 M, about 0.003-about 0.004 M, about 0.004-about 0.005 M, about 0.005-about 0.006 M, or about 0.006-about 0.007 M buffer. In some instances, the buffer is a citrate buffer and the stabilizer includes at least about 0.005 M of the citrate buffer. In some instances, the buffer is citric acid monohydrate and the stabilizer includes at least about 0.005 M of the citric acid monohydrate. In some instances, the buffer is sodium acetate and the stabilizer includes at least about 0.005 M of the sodium acetate. In some instances, the buffer is potassium acetate and the stabilizer includes at least about 0.005 M of the potassium acetate. In some instances, the buffer is sodium phosphate and the stabilizer includes at least about 0.005 M of the sodium phosphate.

In some aspects, a composition of the present disclosure has a pH of from about 3.5 to about 6.5, alternatively about 3.5 to about 4.5, alternatively about 4.5 to about 5, alternatively about 3.5, alternatively about 4, alternatively about 4.5, alternatively about 5, alternatively about 5.5, alternatively about 6.5. In some aspects, compositions of the present disclosure have a pH of from about 6.5 to about 7.5, alternatively about 7.5 to about 8.5. In some aspects, compositions of the present disclosure have a pH of about 8. In some aspects, compositions of the present disclosure have a pH of about 4. In some non-limiting examples, the buffer may be used to adjust the pH.

In some aspects, the non-ionic surfactant is a secondary alcohol ethoxylate, a polyether, a poly (ethylene glycol) derivative, or combinations thereof. In an aspect, the non-ionic surfactant may be Alkyl polyglycoside, Cetomacrogol 1000, Cetostearyl alcohol, Cetyl alcohol, Cocamide DEA, Cocamide MEA, Decyl glucoside, Decyl polyglucose, Glycerol monostearate, IGEPAL CA-630, Isoceteth-20, Lauryl glucoside, Maltoside, Monolaurin, Mycosubtilin, Narrow-range ethoxylate, Nonidet P-40, Nonoxynol-9, Nonoxynols, NP-40, Octaethylene glycol monododecyl ether, N-Octyl beta-D-thioglucopyranoside, Octyl glucoside, Oleyl alcohol, Pentaethylene glycol monododecyl ether, Polidocanol, Poloxamer, Poloxamer 407, Polyethoxylated tallow amine, Polyglycerol polyricinoleate, Polysorbate, Polysorbate 20, Polysorbate 80, Sorbitan, Sorbitan monolaurate, Sorbitan monostearate, Sorbitan tristearate, Stearyl alcohol, Surfactin, Tergitol, Triton X-100, Tween 80, or combinations thereof. In some instances, the stabilizer may include about 0.25 wt. % to about 2.5 wt. % of the non-ionic surfactant, alternatively about 0.5 wt. % to about 2 wt. % of the non-ionic surfactant, alternatively about 0.095 wt. % to about 1.5 wt. % of the non-ionic surfactant. In some instances, the stabilizer may include about 0.10 wt. %, about 0.101 wt. %, about 0.102 wt. %, about 0.103 wt. %, about 0.104 wt. %, about 0.105 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. % or about 1.5 wt. % non-ionic surfactant. In a non-limiting example, the stabilizer includes about 0.095 wt. % to about 0.105 wt. % non-ionic surfactant. In a non-limiting example, the stabilizer includes about 0.1 wt. % Tergitol.

In many instances, calibrators and controls of the present invention are stable while frozen and/or after being thawed. A calibrator (or control) is “stable” for a specified time period under specified conditions if the calibrator (or control) maintains its concentration within a defined range over the specified time period under the specified conditions. In a preferred embodiment, the calibrator is stable if its concentration does not vary by more than 10% (e.g., as determined by an immunoassay) over the specified time period under the specified conditions. In some cases, the calibrator is stable if its concentration does not vary by more than 20% over the specified time period under the specified conditions. For example, a calibrator that does not vary in concentration by more than 10% at frozen temperatures for a period of about 11 months is stable at frozen temperatures for a period of 11 months. Similarly, a calibrator which does not vary in concentration by more than 10% at temperatures from about 2° C.-about 10° C. for a period of about 2 months is stable at about 2° C.-about 10° C. for a period of about 2 months. Various methods of determining calibrator stability are known in the art and generally involve preparing a set of calibrator protein samples at different conditions and measuring their concentrations at different time points using the immunoassay that they are designed to calibrate. As stated, stability is defined as the maximum allowable variation in concentration (e.g., +/−10%) over a particular time period. Further, subjecting the calibrator to stress conditions (e.g., heat and pH extremes) may be used to make a more rapid assessment of stability than would be possible through long-term testing under normal conditions. Subjecting the protein to stress conditions allows for the evaluation of stability over longer time periods because it can accelerate denaturation and degradation of the protein which would normally occur over those longer time periods at normal storage conditions. Such methods of stability testing are well known to skilled artisans (see, e.g., Evaluation of Stability of In Vitro Diagnostic Reagents; Approved Guideline; CLSI document EP25-A. Wayne, PA: Clinical and Laboratory Standards Institute; 2009) and may vary based on the requirements of the immunoassay and the nature of the protein being tested.

The stabilizer is used to stabilize the immunoassay calibrator in the composition. In non-limiting aspects, the immunoassay calibrator is stabilized under frozen storage conditions. Frozen storage conditions mean temperatures that are about −15° C. to about −25° C., alternatively about-18° C., alternatively about −20° C., alternatively about −22° C., alternatively about −24° C. In some aspects, the immunoassay calibrator of the present invention remains stable frozen for at least about 1 month, alternatively at least about 2 months, alternatively at least about 3 months, alternatively at least about 4 months, alternatively at least about 5 months, alternatively at least about 6 months, alternatively at least about 7 months, alternatively at least about 8 months, alternatively at least about 9 months, alternatively at least about 10 months, alternatively at least about 11 months, alternatively at least about 12 months. In one embodiment, the immunoassay calibrator of the present invention remains stable frozen for at least about 11 months. In another embodiment, the immunoassay calibrator of the present invention remains stable frozen for at least about 12 months.

The immunoassay calibrator also remains stable when thawed and kept at temperatures of about 2° C. to about 10° C. This allows a user to keep the composition in a refrigerator for extended periods of time and avoid having to refreeze or lyophilize the composition to maintain the stability of the immunoassay calibrator. In many cases, conventional immunoassay calibrator compositions are generally only stable for short periods of time (typically <24 hours at 2-8° C.). In some aspects, the immunoassay calibrator of the present invention remains stable at temperatures of about 2° C. to about 10° C. for at least about 1 month, alternatively at least about 2 months, alternatively at least about 3 months, alternatively at least about 4 months. In one embodiment, the immunoassay calibrator of the present invention remains stable at temperatures of about 2° C. to about 10° C. for at least about 2 months. In another embodiment, the immunoassay calibrator of the present invention remains stable at temperatures of about 2° C. to about 10° C. for at least about 3 months.

The immunoassay calibrator of the present invention may remain stable through one or more freeze-thaw cycles. Freeze-thaw testing methods for assessing the stability of proteins are well-known in the art and generally involve freezing a protein to a low temperature, thawing the protein to room temperature and then measuring changes in the concentration of the protein or evaluating other indicators of protein denaturation. The immunoassay calibrator of the present invention remains stable after at least one freeze-thaw cycle, alternatively after at least two freeze-thaw cycles, alternatively after at least three freeze-thaw cycles, alternatively after at least four freeze-thaw cycles, alternatively after at least five freeze-thaw cycles. This is a unique advantage of this composition as it eliminates the complexity and requirements of lyophilization while continuing to provide a stable product. In many cases, if a disclosed composition thaws during shipment, it remains useable reducing the requirement for new shipments or reimbursement.

In some embodiments compositions of the present invention comprise an immunoassay calibrator and a stabilizer, wherein the stabilizer comprises a saccharide (e.g., dextran, a dextran derivative, sucrose) and a sugar alcohol, wherein the saccharide and the sugar alcohol are in a weight ratio from about 1:1 to about 5:2; a buffer; and a non-ionic surfactant, wherein the stabilizer is essentially free of BSA, and wherein the immunoassay calibrator is stabilized under frozen storage conditions, and remains stable when thawed and kept refrigerated (e.g., from about 2° C. to about 10° C.) for at least about 1 months or alternatively 2 months. In some instances, the saccharide and the sugar alcohol are in a weight ratio from about 1:1 to about 2:3, alternatively from about 3:1 to about 5:2, alternatively about 1.8:1 to about 2.2:1, alternatively about 1.9:1, alternatively about 2:1, alternatively about 2:3, alternatively about 5:2. In some instances, the saccharide and the sugar alcohol are in a weight ratio from about 2:1. In some instances, the stabilizer comprises about 0.5 wt. % to about 4 wt. % sugar alcohol, alternatively about 1 wt. % to about 3.5 wt. % sugar alcohol, alternatively about 1.5 wt. % to about 2.5 wt. % % sugar alcohol. In some instances, the stabilizer comprises about 0.25 wt. % to about 2.5 wt. % saccharide, alternatively about 0.5 wt. % to about 2 wt. % saccharide, alternatively about 0.5 wt. % to about 1.5 wt. % saccharide. In some instances, the stabilizer comprises at least about 0.005 M buffer. In some instances, the stabilizer comprises about 0.095 wt. % to about 0.105 wt. % non-ionic surfactant. In some instances, the immunoassay calibrator is stabilized under frozen storage conditions for at least about 10 months, at least about 11 months, or at least about 12 months. In some instances, the immunoassay calibrator retains stability after undergoing at least 3, at least 4, or at least 5 freeze thaw cycles. In some instances, the saccharide is DEAE-dextran. In some instances, the sugar alcohol is D-sorbitol.

In some aspects, the disclosed compositions may be used in a method for quantitative immunoassay calibration. An analyte (e.g., a biomarker or diagnostic analyte) contained in a sample may then quantified and/or detected using the calibrated immunoassay. The method of immunoassay calibration may be performed using an immunoassay analyzer (e.g., a high-throughput immunoassay analyzer). Exemplary immunoassay analyzers include ‘Beckman Coulter's Dx19000, Access 2+, UniCel Dx1 600, and UniCel Dx1 800 immunoassay analyzers. The method for calibration may include performing an immunoassay (e.g., an enzyme-linked chemiluminescent immunoassay) on one or more of the compositions disclosed herein and measuring a signal (e.g., a chemiluminescent signal) generated by the one or more compositions. The one or more compositions disclosed herein may having varying, known concentrations of an immunoassay calibrator. For example, the one or more compositions may comprise one, two, three, four, five or six dilutions of an immunoassay calibrator protein or peptide of known concentrations. The method may further comprise plotting the resulting signals against the corresponding known concentrations of the immunoassay calibrator in the one or more compositions. In some instances, using techniques known in the art, a calibration curve may be generated from the plot. For example, to generate the calibration curve, a linear or non-linear regression model may be fit to the data points in the plot. In some instances, the method may further comprise performing the immunoassay on a sample (e.g., a whole blood or plasma sample) containing an analyte (e.g., a disease biomarker). In some instances, the method comprises using the calibration curve to correlate the signal produced by the sample to a concentration of analyte in the sample.

Methods of immunoassay calibration disclosed herein may involve preparing and/or analyzing two or more compositions disclosed herein where each composition has a different concentration of a calibrator protein or peptide disclosed herein. For example, such methods may comprise preparing multiple compositions comprising calibrator proteins in a range of concentrations. In some non-limiting aspects, such methods may include preparing three or more compositions, alternatively four or more compositions, alternatively five or more compositions, or alternatively six or more compositions. Such methods may also comprises analyzing each of the two or more compositions using an immunoassay. In some non-limiting aspects, such methods may include analyzing three or more compositions, alternatively four or more compositions, alternatively five or more compositions, or alternatively six or more compositions. In some instances, the data obtained from the analysis may be used to generate a calibration curve (e.g., a plot of the signal intensity versus the concentration of each calibrator). In some instances, the calibration curve is then used to determine an unknown concentration of a target analyte in a sample.

The disclosed compositions may also be part of a kit. In one aspect, the kit may include two compositions and instructions for use, where the calibrator concentration in the first composition and the second composition are different. The kit may also include a third composition, fourth composition, fifth composition, and/or six composition, where all included compositions have different calibrator concentrations. In some instances, the kit comprises seven compositions and the calibrator is present in the first composition at a concentration of about 0 ng/L, in the second composition at a concentration of about 40-about 80 ng/L, in the third composition at a concentration of about 150-about 250 ng/L, in the fourth composition at a concentration of about 1200 ng/L, in the fifth composition at a concentration of about 2000-about 4500 ng/L, in the sixth composition at a concentration of about 6000-about 13000 ng/L and in the seventh composition at a concentration of about 20,000-about 45,000 ng/L. In some instances, the kit comprises seven compositions and the calibrator is present in the first composition at a concentration of about 0 ng/L, in the second composition at a concentration of about 60 ng/L, in the third composition at a concentration of about 300 ng/L, in the fourth composition at a concentration of about 1200 ng/L, in the fifth composition at a concentration of about 4000 ng/L, in the sixth composition at a concentration of about 12000 ng/L and in the seventh composition at a concentration of about 40,000 ng/L. Depending on the analysis desired, at least one of the compositions in the kit may be a blank. A blank composition is one in which the concentration of the immunoassay calibrator is zero. The kits may further include an antigen of the calibrator. An exemplary antigen is an NT-proBNP or BNP antigen.

### Examples

All reagents, starting materials, and solvents used in the following examples were purchased from commercial suppliers (such as Sigma Aldrich, St. Louis, MO) and were used without further purification unless otherwise indicated.

### Example 1—Freeze-Thaw Stability

A stabilizer was prepared using DEAE dextran (1.0 weight percent), D-Sorbitol (1.0 weight percent), Tergitol (0.1 weight percent), and citric acid monohydrate (0.005 M). The D-Sorbitol and the DEAE-dextran were in a weight ratio of about 2:1. The pH of the stabilizer was about 4.05. The prepared stabilizer was added to different concentrations of NTproBNP, as shown in Table 1, to prepare a calibrator composition. The calibrator composition was then stored at −20° C.

TABLE 1

| | Initial NTproBNP |
|---|---|
| Set # | concentration (ng/L) |
| 1 | 55 |
| 2 | 468 |
| 3 | 3,958 |
| 4 | 21,622 |

A calibrator composition from each set was pulled from the −20° C. and thawed then refrozen. Each composition underwent a total of 7 freeze thaw cycles. After each freeze thaw cycle, an aliquot of the composition was added to a reaction vessel with paramagnetic particles coated with an antibody directed towards NTproBNP. After incubation in a reaction vessel, materials bound to the paramagnetic particles were held in a magnetic field, while unbound materials were washed away. A chemiluminescent substrate was added to the reaction vessel and the light generation by the reaction was measured using a luminometer.

As shown in Table 2 the calibrator's concentration was equal to or substantially similar to (±10%) the initial concentration at the end of the freeze-thaw cycles.

TABLE 2

| | Final NTproBNP | |
|---|---|---|
| Set # | concentration (ng/L) | % change |
| 1 | 56.4 | +1.89% |
| 2 | 469.8 | +0.38% |
| 3 | 3,968.1 | +0.25% |
| 4 | 21,822.8 | +0.93% |

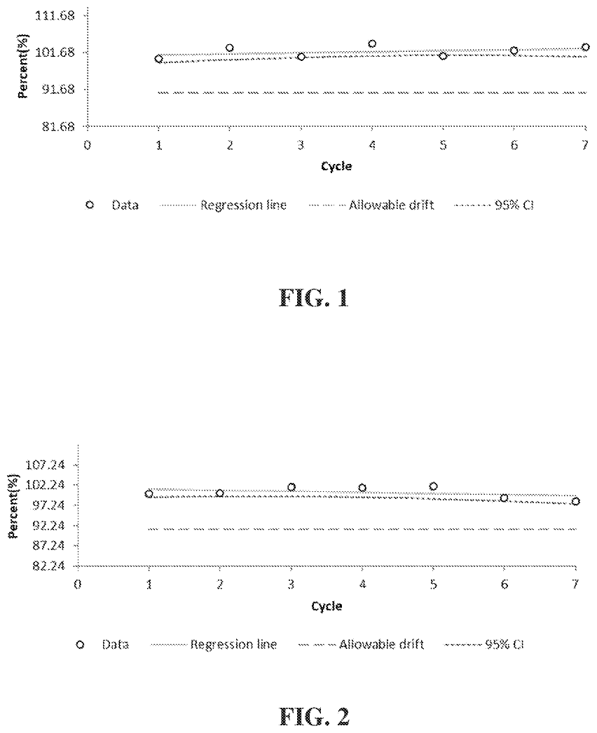

FIG. 1 depicts a linear model for percent (%) change in calibrator concentration versus freeze-thaw cycle for a calibrator composition containing 55 ng/L of NTproBNP. As shown in FIG. 1, each data point corresponds to a freeze thaw cycle, i.e., point 1 is an aliquot analyzed after the first freeze-thaw cycle, point 2 is an aliquot analyzed after the second freeze-thaw cycle, etc. The regression line shows the linear relationship between each data point and the observed concentration variation. As shown in FIG. 1 (as well as FIGS. 2-4), the slope of the regression line is near zero or substantially zero. Substantially zero indicates a slope between about-0.5% to about +0.5%. A slope that falls outside of this range may indicate that the calibrator composition is unstable.

The 95% Cl is the lower confidence limit, indicating that the margin of error for the calculation of the concentration of the data point is small. As such, it is expected that these results are reproducible with only a small margin (5%) of calculated values potentially falling below the 95% Cl line.

The allowable drift limit was set at −10%. If a data point were to fall below the lower limit of the allowable drift line (e.g., appx 91% in FIG. 1), then the calibrator composition would be deemed unstable. As shown in FIG. 1 (and FIGS. 2-4), all of the data points are above the allowable drift line. Additionally, the regression line and 95% Cl line are above the allowable drift line, further supporting the finding of stability of the calibrator composition after multiple freeze-thaw cycles.

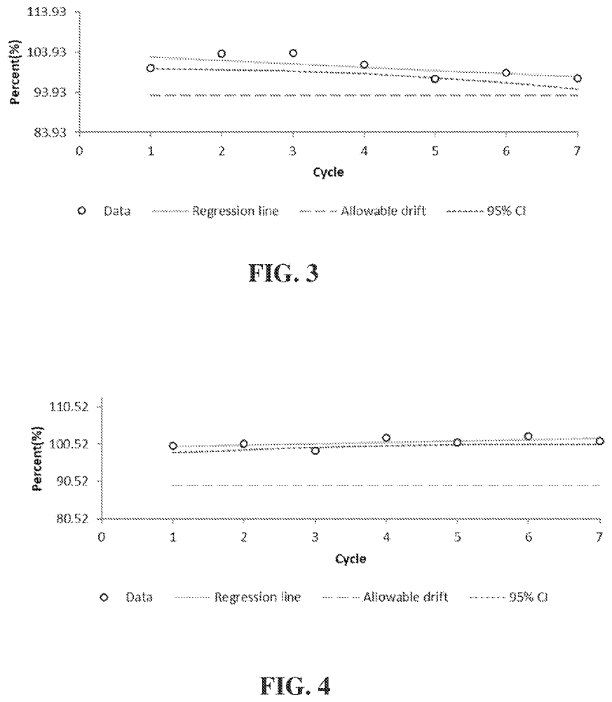

FIG. 2 depicts a linear model for percent (%) change in calibrator concentration versus freeze-thaw cycle for a calibrator composition containing 468 ng/L of NTproBNP. FIG. 3 depicts a linear model for percent (%) change in calibrator concentration versus freeze-thaw cycle for a calibrator composition containing 3,958 ng/L of NTproBNP. FIG. 4 depicts a linear model for percent (%) change in calibrator concentration versus freeze-thaw cycle for a calibrator composition containing 21,622 ng/L of NTproBNP.

### Example 2—Short Term Stability Analysis

A stabilizer was prepared using the procedures of Example 1. The prepared stabilizer was added to different concentrations of NTproBNP, as shown in Table 3, to prepare a calibrator composition. The calibrator composition was then stored at appx −15° C. to −20° C.

TABLE 3

| | NTproBNP concentration |
|---|---|
| Set # | (ng/L) |
| 1 | 60 |
| 2 | 200 |
| 3 | 650 |
| 4 | 2200 |
| 5 | 7000 |
| 6 | 23000 |

To test short-term stability, the calibrator was thawed and kept refrigerated (e.g., from about 2° C. to about 10° C.) and evaluated at several different time points (0 days, 1 day, 13 days, 26 days, 39 days, 52 days, and 65 days). An aliquot of the composition at each time point was added to a reaction vessel with paramagnetic particles coated with an antibody directed towards NTproBNP. After incubation in a reaction vessel, materials bound to the paramagnetic particles were held in a magnetic field, while unbound materials were washed away. A chemiluminescent substrate was added to the reaction vessel and the light generation by the reaction was measured using a luminometer.

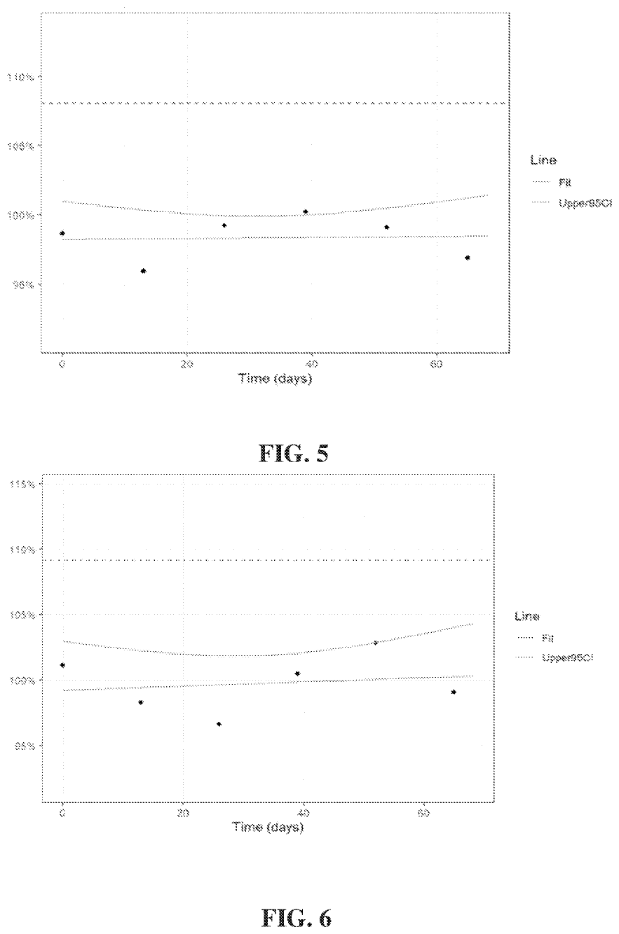

As shown in FIGS. 5-10, at each time point the calibrator's concentration was equal to or substantially similar to (±10%) the concentration of a reference.

FIG. 5 depicts a linear model of short-term stability for calibrator composition containing 60 ng/L of NT-proBNP and stored at refrigerated temperatures. As shown in FIG. 5, each data point corresponds to the day the aliquot was evaluated. The fit line shows the linear relationship between each data point and the observed concentration variation. As shown in FIG. 5 (as well as FIGS. 6-10), the slope of the regression line is zero or substantially zero.

The Upper95 Cl is the upper confidence limit, indicating that the margin of error for the calculation of the concentration of the data point is small. As such, it is expected that these results are reproducible with only a small margin (5%) of calculated values potentially falling above the 95% Cl line. The dashed line corresponds to the allowable drift, which was set at +10%. If a data point were to exceed limit of the allowable drift line (e.g., appx. 108% in FIG. 5), then the calibrator composition would be deemed unstable. As shown in FIG. 5 (and FIGS. 6-10), all of the data points are below the allowable drift line. Additionally, the fit line and Upper95 Cl line are below the allowable drift line, further supporting the finding of short-term stability of the calibrator composition.

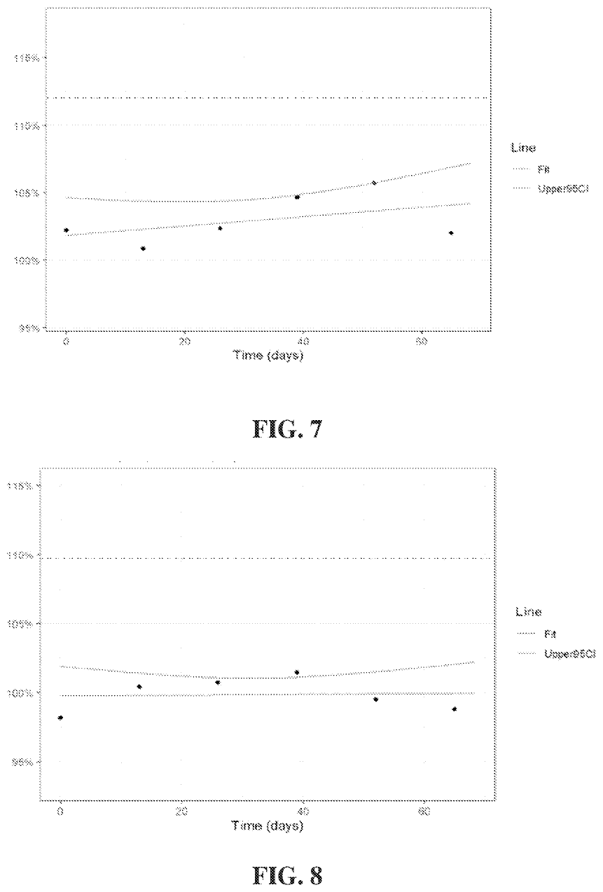

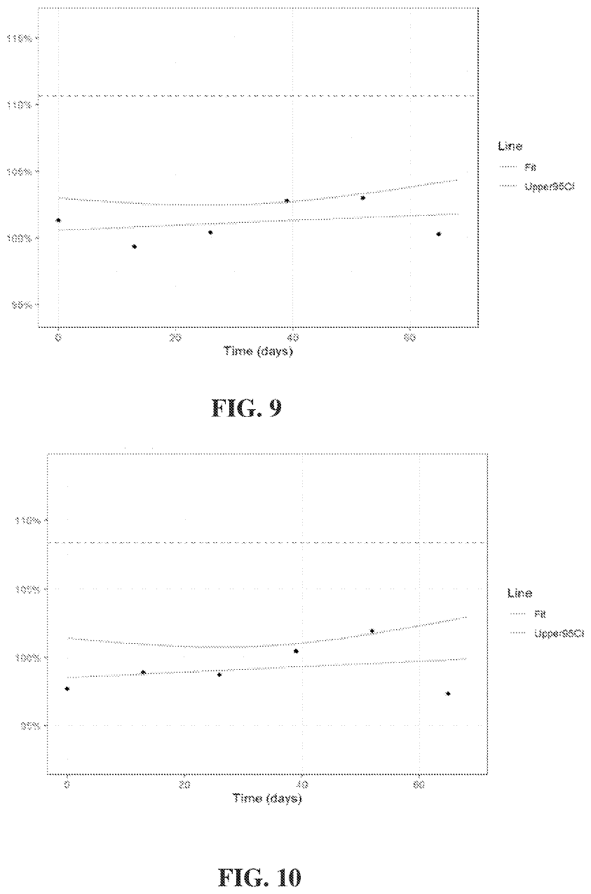

FIG. 6 depicts a linear model of short-term stability for a calibrator composition containing 200 ng/L of NT-proBNP and stored at refrigerated temperatures. FIG. 7 depicts a linear model of short-term stability for a calibrator composition containing 650 ng/L of NT-proBNP and stored at refrigerated temperatures. FIG. 8 depicts a linear model of short-term stability for a calibrator composition containing 2200 ng/L of NT-proBNP and stored at refrigerated temperatures. FIG. 9 depicts a linear model of short-term stability for a calibrator composition containing 7000 ng/L of NT-proBNP and stored at refrigerated temperatures. FIG. 10 depicts a linear model of short-term stability for a calibrator composition containing 23000 ng/L of NT-proBNP and stored at refrigerated temperatures.

### Example 3—Long-Term Stability Analysis

A stabilizer was prepared using the procedures of Example 1. The prepared stabilizer was added to different concentrations of NTproBNP, as shown in Table 3, to prepare a calibrator composition. The calibrator composition was then stored at appx −15° C. to −20° C.

To test long term stability, the calibrator was evaluated at several different time points (0 days, 61 days, 110 days, 161 days, 216 days, 273 days, 315 days, 349 days, 372 days, 419 days, and 420 days) using the methods of Example 2. At each testing time point, an aliquot of the calibrator composition was thawed and kept refrigerated (e.g., from about 2° C. to about 10° C.) prior to analysis and replicate measurements were taken.

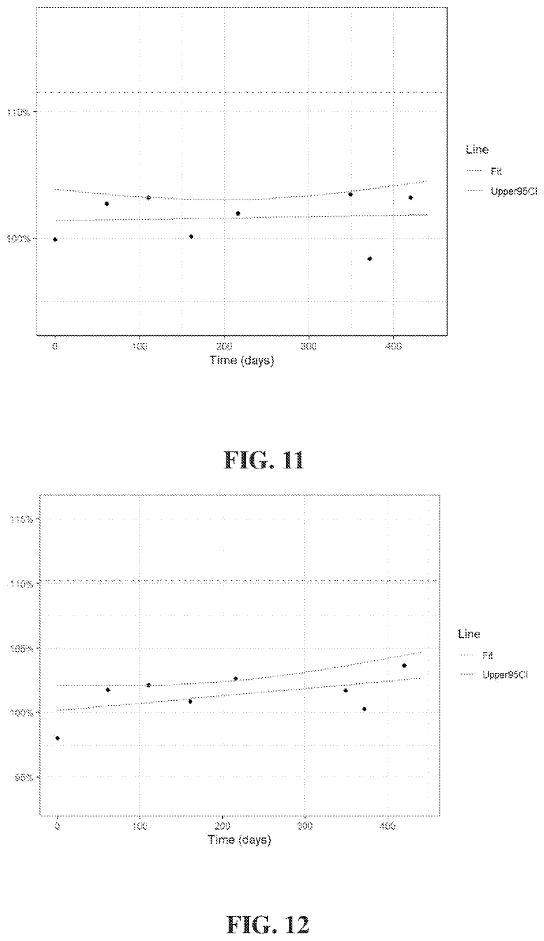

As shown in FIGS. 11-16, at each time point the calibrator's concentration was equal to or substantially similar to (±10%) the concentration of a reference.

FIG. 11 depicts a linear model of long-term stability for calibrator composition containing 60 ng/L of NT-proBNP and stored at frozen temperatures. As shown in FIG. 11, each data point corresponds to the day the aliquot was evaluated. The fit line shows the linear relationship between each data point and the observed concentration variation. As shown in FIG. 11 (as well as FIGS. 12-16), the slope of the regression line is zero or substantially zero.

The Upper95 Cl is the upper confidence limit, indicating that the margin of error for the calculation of the concentration of the data point is small. As such, it is expected that these results are reproducible with only a small margin (5%) of calculated values potentially falling above the 95% Cl line. The dashed line corresponds to the allowable drift, which was set at +10%. If a data point were to exceed limit of the allowable drift line (e.g., appx. 110% in FIG. 11), then the calibrator composition would be deemed unstable. As shown in FIG. 11 (and FIGS. 12-16), all of the data points are below the allowable drift line. Additionally, the fit line and Upper95 Cl line are below the allowable drift line, further supporting the finding of long-term stability of the calibrator composition.

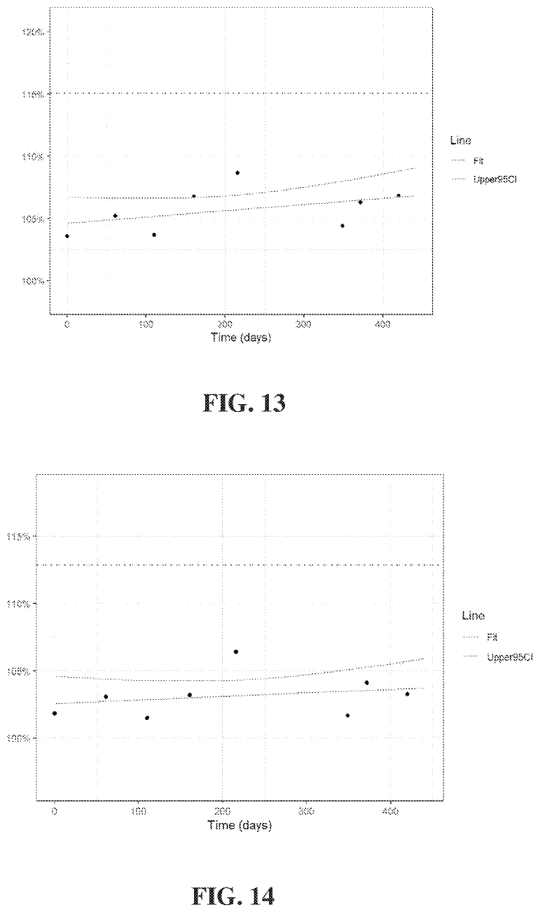

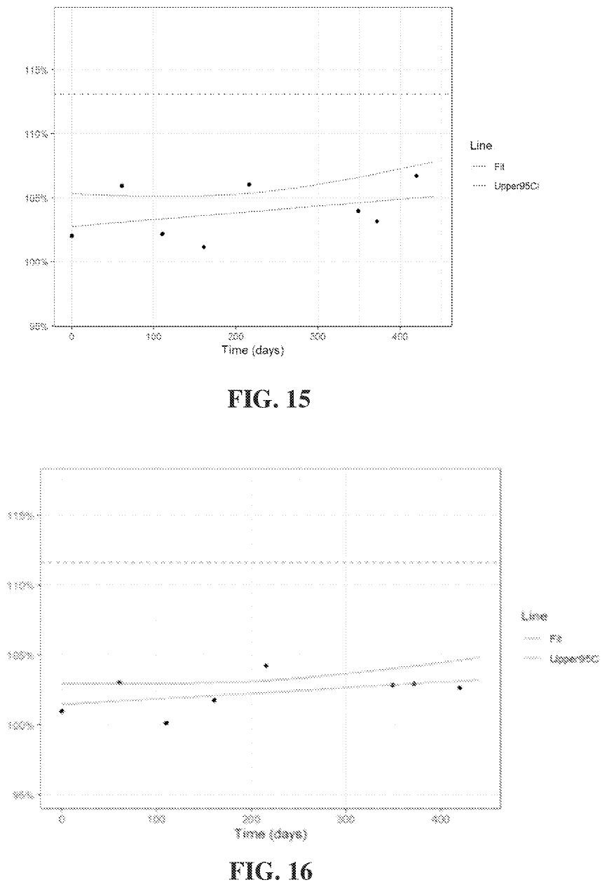

FIG. 12 depicts a linear model of long-term stability for a calibrator composition containing 200 ng/L of NT-proBNP and stored at frozen temperatures. FIG. 13 depicts a linear model of long-term stability for a calibrator composition containing 650 ng/L of NT-proBNP and stored at frozen temperatures. FIG. 14 depicts a linear model of long-term stability for a calibrator composition containing 2200 ng/L of NT-proBNP. FIG. 15 depicts a linear model of long-term stability for a calibrator composition containing 7000 ng/L of NT-proBNP and stored at frozen temperatures. FIG. 16 depicts a linear model of long-term stability for a calibrator composition containing 23000 ng/L of NT-proBNP and stored at frozen temperatures.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern.

It will be understood that while the invention has been described in conjunction with the detailed description thereof, the foregoing description is intended to illustrate and not limit the scope of the invention, which is defined by the scope of the appended claims. The foregoing detailed description and examples have been given for clarity of understanding only. No unnecessary limitations are to be understood therefrom. The invention is not limited to the exact details shown and described, for variations obvious to one skilled in the art will be included within the invention defined by the claims. Other aspects, advantages, and modifications are within the scope of the following claims.

What is claimed is:

1. A composition comprising:
at least one calibrator; and
a stabilizer, wherein the stabilizer comprises
Diethylaminoethyl-(“DEAE”) dextran;
D-Sorbitol, wherein the D-Sorbitol and DEAE-dextran are in a weight ratio from about 1:1 to about 5:2,
a buffer; and
a non-ionic surfactant;
wherein the stabilizer is essentially free of bovine serum albumin; and
wherein the calibrator is stabilized under frozen storage conditions, and remains stable when thawed and kept at temperatures of from about 2° C. to about 10° C.

2. The composition of claim 1, wherein the calibrator remains stable under frozen conditions for at least about 1 month, alternatively at least about 2 months, alternatively at least about 3 months, alternatively at least about 4 months, alternatively at least about 5 months, alternatively at least about 6 months, alternatively at least about 7 months, alternatively at least about 8 months, alternatively at least about 9 months, alternatively at least about 10 months, alternatively at least about 11 months, alternatively at least about 12 months.

3. The composition of claim 1 or claim 2, wherein the calibrator remains stable when thawed and kept at temperatures of from about 2° C. to about 10° C. for at least about 24 hours, alternatively at least about 3 days, alternatively at least about 1 week, alternatively at least about 2 weeks, alternatively at least about 3 weeks, alternatively at least about 4 weeks, alternatively at least about 5 weeks, alternatively at least about 6 weeks, alternatively at least about 7 weeks, alternatively at least about 8 weeks, alternatively at least about 9 weeks.

4. The composition of any one of the preceding claims, wherein the calibrator remains stable after at least one freeze-thaw cycle, alternatively after at least two freeze-thaw cycles, alternatively after at least three freeze-thaw cycles, alternatively after at least four freeze-thaw cycles, alternatively after at least five freeze-thaw cycles, alternatively after at least six freeze-thaw cycles, alternatively after at least seven freeze-thaw cycles.

5. The composition of any one of the preceding claims, wherein the D-Sorbitol and DEAE-dextran are in a weight ratio from about 1:1 to about 2:3, alternatively from about 3:1 to about 5:2, alternatively from about 1.8:1 to about 2.2:1, alternatively about 1.9:1, alternatively about 2:1, alternatively about 2:3, alternatively about 5:2.

6. The composition of any one of the preceding claims, wherein the stabilizer comprises about 0.5 wt. % to about 4 wt. % D-Sorbitol, alternatively about 1 wt. % to about 3.5 wt. % D-Sorbitol, alternatively about 1.5 wt. % to about 2.5 wt. % D-Sorbitol, alternatively about 1.6 wt. % D-Sorbitol, alternatively about 1.7 wt. % D-Sorbitol, alternatively about 1.8 wt. % D-Sorbitol, alternatively about 1.9 wt. % D-Sorbitol, alternatively about 2.0 wt. % D-Sorbitol, alternatively about 2.1 wt. % D-Sorbitol, alternatively about 2.2 wt. % D-Sorbitol, alternatively about 2.3 wt. % D-Sorbitol, alternatively about 2.4 wt. % D-Sorbitol, or alternatively about 2.5 wt. % D-Sorbitol.

7. The composition of any one of the preceding claims, wherein the stabilizer comprises about 0.25 wt. % to about 2.5 wt. % DEAE-dextran, alternatively about 0.5 wt. % to about 2 wt. % DEAE-dextran, alternatively about 0.5 wt. % to about 1.5 wt. % DEAE-dextran, alternatively about 0.6 wt. % DEAE-dextran, alternatively about 0.7 wt. % DEAE-dextran, alternatively about 0.8 wt. % DEAE-dextran, alternatively about 0.9 wt. % DEAE-dextran, alternatively about 1.0 wt. % DEAE-dextran, alternatively about 1.1 wt. % DEAE-dextran, alternatively about 1.2 wt. % DEAE-dextran, alternatively about 1.3 wt. % DEAE-dextran, or alternatively about 1.4 wt. % DEAE-dextran.

8. The composition of any one of the preceding claims, wherein the buffer is a citrate buffer, an acetate buffer, a phosphate buffer, or combinations thereof.

9. The composition of claim 8, wherein the buffer is selected from the group consisting of citrate, citric acid monohydrate, sodium acetate, potassium acetate, sodium phosphate, or combinations thereof.

10. The composition of any one of the preceding claims, wherein the stabilizer comprises at least about 0.0025 M buffer, alternatively at least about 0.001 M buffer, alternatively at least about 0.002 M buffer, alternatively at least about 0.003 M buffer, alternatively at least about 0.004 M buffer, alternatively at least about 0.005 M buffer, alternatively at least about 0.006 M buffer, or alternatively at least about 0.007 M buffer.

11. The composition of any one of the preceding claims, wherein the non-ionic surfactant is a secondary alcohol ethoxylate, a polyether, a poly(ethylene glycol) derivative, or combinations thereof.

12. The composition of any one of the preceding claims, wherein the non-ionic surfactant is selected from the group consisting of Alkyl polyglycoside, Cetomacrogol 1000, Cetostearyl alcohol, Cetyl alcohol, Cocamide DEA, Cocamide MEA, Decyl glucoside, Decyl polyglucose, Glycerol monostearate, IGEPAL CA-630, Isoceteth-20, Lauryl glucoside, Maltoside, Monolaurin, Mycosubtilin, Narrow-range ethoxylate, Nonidet P-40, Nonoxynol-9, Nonoxynols, NP-40, Octaethylene glycol monododecyl ether, N-Octyl beta-D-thioglucopyranoside, Octyl glucoside, Oleyl alcohol, Pentaethylene glycol monododecyl ether, Polidocanol, Poloxamer, Poloxamer 407, Polyethoxylated tallow amine, Polyglycerol polyricinoleate, Polysorbate, Polysorbate 20, Polysorbate 80, Sorbitan, Sorbitan monolaurate, Sorbitan monostearate, Sorbitan tristearate, Stearyl alcohol, Surfactin, Tergitol, Triton X-100, Tween 80, and combinations thereof.

13. The composition of any one of the preceding claims, wherein the stabilizer comprises about 0.25 wt. % to about 2.5 wt. % of the non-ionic surfactant, alternatively about 0.5 wt. % to about 2 wt. % of the non-ionic surfactant, alternatively about 0.095 wt. % to about 1.5 wt. % of the non-ionic surfactant, alternatively about 0.10 wt. % of the non-ionic surfactant, alternatively about 0.101 wt. % of the non-ionic surfactant, alternatively about 0.102 wt. % of the non-ionic surfactant, alternatively about 0.103 wt. % of the non-ionic surfactant, alternatively about 0.104 wt. % of the non-ionic surfactant, alternatively about 0.105 wt. % of the non-ionic surfactant, alternatively about 0.2 wt. % of the non-ionic surfactant, alternatively about 0.3 wt. % of the non-ionic surfactant, alternatively about 0.4 wt. % of the non-ionic surfactant, alternatively about 0.5 wt. % of the non-ionic surfactant, alternatively about 0.6 wt. % of the non-ionic surfactant, alternatively about 0.7 wt. % of the non-ionic surfactant, alternatively about 0.8 wt. % of the non-ionic surfactant, alternatively about 0.9 wt. % of the non-ionic surfactant, alternatively about 1.0 wt. % of the non-ionic surfactant, alternatively about 1.1 wt. % of the non-ionic surfactant, alternatively about 1.2 wt. % of the non-ionic surfactant, alternatively about 1.3 wt. % of the non-ionic surfactant, alternatively about 1.4 wt. % of the non-ionic surfactant, alternatively about 1.5 wt. % of the non-ionic surfactant, or alternatively about 0.095 wt. % to about 0.105 wt. % non-ionic surfactant.

14. The composition of any one of the preceding claims, wherein the stabilizer is free of bovine serum albumin.

15. The composition of any one of the preceding claims, wherein the composition is essentially free of bovine serum albumin, alternatively free of bovine serum albumin.

16. The composition of any one of the preceding claims, wherein the composition is essentially free of protease inhibitors, preferably free of protease inhibitors.

17. The composition of any one of the preceding claims, wherein the composition is a liquid or a solution.

18. The composition of any one of the preceding claims, wherein the composition has a pH of from about 3.5 to about 6.5, alternatively about 3.5 to about 4.5, alternatively about 4.5 to about 5, alternatively about 3.5, alternatively about 4, alternatively about 4.5, alternatively about 5, alternatively about 5.5, alternatively about 6.5.

19. The composition of any one of the preceding claims, wherein the calibrator is an immunoassay calibrator.

20. The composition of any one of the preceding claims, wherein the calibrator is substantially similar to a target analyte.

21. The composition of claim 20, wherein the target analyte is a biomarker or a diagnostic analyte.

22. The composition of any one of the preceding claims, wherein the calibrator is a protein, peptide, or amino acid.

23. The composition of any one of the preceding claims, wherein the calibrator is recombinantly or synthetically produced.

24. The composition of any one of the preceding claims, wherein the calibrator comprises a post-translational modification.

25. The composition of claim 24, wherein the post-translational modification is glycosylation, phosphorylation, or acetylation.

26. The composition of any one of claims 22 to 25, wherein the calibrator is a natriuretic peptide or natriuretic protein.

27. The composition of claim 26, wherein the calibrator is a recombinantly produced and glycosylated N-Terminal Pro-B-Type Natriuretic peptide (NT-proBNP).

28. The composition of claim 26, wherein the calibrator is a synthetically produced NT-proBNP.

29. The composition of claim 26, wherein the calibrator is a recombinant B-type natriuretic peptide (BNP).

30. A method for quantitative assay calibration, the method comprising:
analyzing at least one composition of the preceding claims using an immunoassay,
wherein the analyzing comprises detecting the presence, absence, and/or quantity of the calibrator in the at least one composition, and generating a corresponding set of values; and
wherein the corresponding set of values is used to generate a calibration curve.

31. The method of claim 30, wherein the method comprises analyzing at least two compositions of any one of the preceding claims; alternatively at least three compositions of any one of the preceding claims; alternatively at least four compositions of any one of the preceding claims; alternatively at least five compositions of any one of the preceding claims; alternatively at least six compositions of any one of the preceding claims and wherein each composition comprises a different concentration of the calibrator.

32. The method of claim 30 or claim 31, wherein the calibration curve a non-linear calibration curve.

33. The method of claim 32, wherein the non-linear calibration curve is used to quantify a target analyte.

34. The method of claim 32 or claim 33, wherein the non-linear calibration curve comprises at least two values, wherein one value corresponds to the concentration of calibrator in the first composition and one value corresponds to the concentration of calibrator in the second composition.

35. The method of claim 34, wherein the non-linear calibration curve comprises at least three values, wherein one value corresponds to the concentration of calibrator in the third composition.

36. The method of claim 35, wherein the non-linear calibration curve comprises at least four values, wherein one value corresponds to the concentration of calibrator in the fourth composition.

37. The method of claim 36, wherein the non-linear calibration curve comprises at least five values, wherein one value corresponds to the concentration of calibrator in the fifth composition.

38. The method of claim 37, wherein the non-linear calibration curve comprises at least six values, wherein one value corresponds to the concentration of calibrator in the sixth composition.

39. The method of any one of claims 30 to 38, wherein the method is conducted using an analyzer.

40. The method of claim 39, wherein the analyzer is an immunoassay analyzer.

41. The method of claim 39 or claim 40, wherein the analyzer is a high-throughput analyzer.

42. The method of any one of claims 30 to 41, wherein the calibrator is a natriuretic peptide or natriuretic protein.

43. The method of claim 42, wherein the calibrator is a recombinantly produced and glycosylated N-Terminal Pro-B-Type Natriuretic peptide (NT-proBNP).

44. The method of claim 42, wherein the calibrator is a synthetically produced NT-proBNP.

45. The method of claim 42, wherein the calibrator is a recombinant B-type natriuretic peptide (BNP).

46. A kit comprising at least a first composition according to any one of the preceding claims and a second composition according to any one of the preceding claims and instructions for use, wherein the immunoassay calibrator concentration in the first composition and the second composition are different.

47. The kit of claim 46, further comprising a third composition according to any one of the preceding claims, wherein the immunoassay calibrator concentration in the third composition is different from the immunoassay calibrator concentration in the first composition and the second composition.

48. The kit of claim 47, further comprising a fourth composition according to any one of the preceding claims, wherein the immunoassay calibrator concentration in the fourth composition is different from the immunoassay calibrator concentration in the first composition, the second composition, and the third composition.

49. The kit of claim 48, further comprising a fifth composition according to any one of the preceding claims, wherein the immunoassay calibrator concentration in the fifth composition is different from the immunoassay calibrator concentration in the first composition, the second composition, the third composition, and the fourth composition.

50. The kit of claim 49, further comprising a sixth composition according to any one of the preceding claims, wherein the calibrator concentration in the sixth composition is different from the calibrator concentration in the first composition, the second composition, the third composition, the fourth composition, and the fifth composition.

51. The kit of any one of claims 46 to 50, wherein the calibrator concentration in the third composition, fourth composition, fifth composition, or sixth composition is zero.

52. The kit of any one of claims 46 to 51, wherein the first composition, second composition, third composition, fourth composition, fifth composition, and/or the sixth composition are in separate units, vials, or containers.

53. The kit of any one of claims 46 to 52, further comprising an antigen of the calibrator.

54. The kit of claim 53, wherein the antigen is an NT-proBNP or BNP antigen.
